(19) United States
(12) Patent Application Publication
(10) Pub. No.: US 2026/0251737 A1
(43) Pub. Date: Aug. 27, 2026

(54) TORSO LOCAL COIL ARRAY

(71) Applicant: Siemens Healthineers AG, Forchheim (DE)

(72) Inventors: Ralph Kimmlingen, Zirndorf (DE); Martin Schröder, Möhrendorf (DE)

(21) Appl. No.: 19/552,007

(22) Filed: Feb. 27, 2026

(30) Foreign Application Priority Data
Feb. 27, 2025 (EP) 25160459

(51) Int. Cl. G01R 33/3415

(52) U.S. Cl. CPC G01R 33/3415

(57) ABSTRACT

A local coil array, a patient table, and a magnetic resonance apparatus are provided. The local coil array includes an anterior part that comprises a bearing structure and an anterior radio-frequency transmitting unit. The bearing structure has a supporting side and an upper side opposite the supporting side. The anterior radio-frequency transmitting unit is arranged on the upper side of the bearing structure. The bearing structure has an inner volume configured to position the section of torso of the patient therein.

This application claims the benefit of European Patent Application No. EP 25160459, filed on Feb. 27, 2025, which is hereby incorporated by reference in its entirety.

### BACKGROUND

The present embodiments relate to a local coil array, a patient table, and a magnetic resonance apparatus.

In medical engineering, imaging by magnetic resonance (MR), also referred to as magnetic resonance imaging (MRI), is characterized by high soft tissue contrasts. In this connection, radio-frequency (RF) signals (e.g., pulses) for generating an RF field in an examination region, in which a patient to be examined is located, are irradiated with the aid of a magnetic resonance apparatus during a magnetic resonance measurement. As a result, spatially encoded magnetic resonance signals are triggered in the patient. The magnetic resonance signals are received by the magnetic resonance apparatus and used to reconstruct magnetic resonance mappings.

In order to excite signals in magnetic resonance apparatuses with ultra-high magnetic fields, which amount, for example, to at least 7 T, combined RF transmit-receive local coil arrays are often employed. Thus, for example, fixed local coil arrays that offer optimized transmit and receive efficiency for a specific body region (e.g., head coils) are known. An important criterion for the clinical application of such coils is simple but, at the same time, precise handling operation in order to provide, for example, that predefined SAR limit values are not overshot.

Freely positionable local coil arrays are also known, which may be attached to the magnetic resonance apparatus by freely movable cables. Similarly, the use of positioning aids, such as tie-down straps, is known. However, such solutions do not enable a standardized clinical workflow; rather, such solutions are better suited to research applications in which the local coil array may be individually adjusted to the examination.

### SUMMARY AND DESCRIPTION

The scope of the present invention is defined solely by the appended claims and is not affected to any degree by the statements within this summary.

The present embodiments may obviate one or more of the drawbacks or limitations in the related art. For example, a local coil array for transmitting (and optionally also receiving) RF signals that improves the clinical workflow is provided.

Accordingly, a local coil array for the magnetic resonance examination of a section of a torso of a patient (e.g., human patient) is provided. The local coil array includes an anterior part that includes a bearing structure and an anterior radio-frequency transmitting unit. The bearing structure has a supporting side and an upper side (e.g., opposite the supporting side). The anterior radio-frequency transmitting unit is arranged on the upper side of the bearing structure. The bearing structure has an inner volume configured to position the section of torso of the patient therein.

The torso customarily includes the chest, abdomen, back and pelvis of the patient. The torso may also be referred to as the torso. For example, the torso (within the meaning of this disclosure) may be the region of the body above the knee and below the neck of the patient. The section of torso to be examined is a section of the torso. The section of torso may include part of the torso or the entire torso. In the examination, using the local coil array, it is therefore not imperative for the entire torso to be examined; rather, the examination may also relate to only a section of the torso. Similarly, it is not imperative for the entire torso to be positioned in the inner volume of the bearing structure; rather, if necessary, only a section of the torso may be positioned in the inner volume of the bearing structure.

The anterior part is, for example, an upper part and/or a top part. When the local coil array is used as intended, the anterior part is, for example, arranged wholly or partially above the section of torso of the patient. Here and below, intended use of the local coil array may be, for example, the use of the local coil array during a magnetic resonance measurement. In one embodiment, the local coil array is located in an examination region of a magnetic resonance apparatus and/or on a patient table of the magnetic resonance apparatus during the magnetic resonance measurement. For example, the patient is supported on the patient table during the magnetic resonance measurement.

The anterior radio-frequency transmitting unit is configured, for example, to transmit RF signals. For example, nuclear spins may be excited in the patient (e.g., in the torso of the patient) by the transmitted RF signals. A relaxation of the nuclear spin magnetization may generate magnetic resonance signals that may be acquired by the magnetic resonance apparatus as measurement data.

The anterior radio-frequency transmitting unit may include, for example, one or more transmitting antennas. For example, each transmitting antenna may be assigned to a transmission channel. If a plurality of transmitting antennas or transmission channels is present, a parallel transmission (pTx) may be possible with the local coil array.

In one embodiment, the local coil array, apart from one or more radio-frequency transmitting units, such as, for example, the anterior radio-frequency transmitting unit, also includes one or more radio-frequency transmitting units for receiving RF signals (e.g., magnetic resonance signals).

For example, the supporting side of the bearing structure is configured to be arranged on a support. For example, the supporting side of the bearing structure is the lower side of the bearing structure. For example, the bearing structure may be placed on the support with the supporting side downwards.

The bearing structure may have one or more supporting surfaces and/or support points on the supporting side of the bearing structure. In one embodiment, these supporting surfaces and/or support points touch the support when the bearing structure is arranged on the support.

The bearing structure may be rigid. In one embodiment, the bearing structure is configured to bear and/or support and/or receive the anterior radio-frequency transmitting unit. The bearing structure may be composed of one or more individual parts. The material of the bearing structure may include, for example, a plastics material (e.g., glass reinforced plastic (GRP) and/or polyvinyl chloride (PVC)). In one embodiment, the rigid bearing structure provides that the anterior radio-frequency transmitting unit does not simply rest in only an undefined manner on the body of the patient; rather, the anterior radio-frequency transmitting unit may be positioned in a defined manner relative to the patient.

In one embodiment, the inner volume of the bearing structure is located between the supporting side and the upper side of the bearing structure. In one embodiment, the supporting side and the upper side have a spacing from one another, which is large enough to be able to position the section of torso of the patient in between. In one embodiment, RF signals may be irradiated from above into the section of torso by the anterior radio-frequency transmitting unit.

For example, the bearing structure delimits the inner volume. In one embodiment, the inner volume of the bearing structure is configured to be open at least in one direction. In one embodiment, the inner volume of the bearing structure is configured to be open at two opposite points (e.g., ends) of the bearing structure. In one embodiment, the inner volume of the bearing structure is configured to be open perpendicularly to the transversal plane. In one embodiment, the inner volume of the bearing structure is delimited by a foot-end opening and/or by a head-end opening perpendicular to the transversal plane. In one embodiment, the inner volume of the bearing structure is configured to be open such that when the local coil array is used, a foot-end portion of the body (e.g., the lower body) of the patient extends outwards through a foot-end opening of the inner volume, and/or a head-end portion of the body (e.g., the head) of the patient extends outwards through a head-end opening of the inner volume.

The patient may be assumed to be an adult human patient (e.g., with a height of 150 to 200 cm).

The transversal plane may refer to the anatomical plane of a (e.g., typical) body of the (e.g., human) patient when its section of torso is positioned (e.g., as intended) in the local coil array. This applies equally to the terms “sagittal plane” and “frontal plane,” which are used below. Even though these terms refer first to the anatomy of the patient, these terms may be transferred to the proposed local coil array for the characterization thereof and/or be used (e.g., analogously) for this in that an intended positioning of the patient in the local coil array is assumed. The transversal plane and/or the sagittal plane and/or the frontal plane correspond, for example, to the respective anatomical plane of the body of the patient when this is positioned as intended in the local coil array. For example, the frontal plane and/or sagittal plane and/or the transversal plane refer to the anatomy of the patient when the anatomy is positioned as intended in the local coil array. Intended positioning may exist when the chest or the back of the patient faces the upper side of the bearing structure and/or the anterior radio-frequency transmitting unit.

A transversal plane customarily denotes a plane that is oriented perpendicularly to the longitudinal axis of the patient. The longitudinal axis of the patient customarily connects head and feet of the patient.

The sagittal plane customarily denotes a plane that divides the body into a right half and a left half of the body. For example, the right half of the body includes the right leg, and the left half of the body includes the left leg of the patient. The sagittal plane is oriented perpendicularly to the transversal plane.

The frontal plane customarily denotes a plane that divides the body into a front portion and a back portion. For example, the front portion includes the abdomen of the patient, and the back portion includes the patient's back. The frontal plane is oriented perpendicularly to the transversal plane and/or perpendicularly to the sagittal plane. For example, the front portion of the patient faces the upper side of the bearing structure. A plane arranged centrally in the local coil array and which is oriented parallel to the frontal plane separates the supporting side of the bearing structure from the upper side of the bearing structure.

In one embodiment, the inner volume of the bearing structure has a length, perpendicular to the transversal plane, between 50 to 80 cm (e.g., 60 to 70 cm). In one embodiment, the inner volume has a width, perpendicular to the sagittal plane, between 40 to 70 cm (e.g., between 50 to 60 cm). In one embodiment, the inner volume has a width, perpendicular to the frontal plane, between 30 to 60 cm (e.g., between 40 to 50 cm).

In one embodiment, the supporting side of the bearing structure and/or the upper side of the bearing structure are oriented parallel to a frontal plane of the patient.

In one embodiment, the bearing structure has two lateral sections that extend parallel to the sagittal plane. In one embodiment, the inner volume of the bearing structure is delimited perpendicular to the sagittal plane by the two sides lateral sections.

The bearing structure (e.g., lateral sections of the bearing structure) has, for example, in a side view onto the sagittal plane, a C-shape (e.g., a shape of a C-arm). In one embodiment, the viewing direction of the side view onto the sagittal plane is oriented perpendicular to the sagittal plane. For example, a right lateral section and a left lateral section, respectively, of the bearing structure may have the C-shape, so reference may be made, for example, to a double C-arm.

In one embodiment, the C-shape of the bearing structure is characterized by an (e.g., elongate) recess that is suitable for positioning an arm (e.g., upper arm, lower arm, and/or hand) and/or a shoulder of the patient at least partially therein. In one embodiment, the patient has more space laterally as a result of the recess. In one embodiment, the comfort of the patient during a magnetic resonance examination with the local coil array may thus be increased.

In one embodiment, the local coil array includes an anti-trap structure that is arranged on the recess. In one embodiment, the anti-trap structure may prevent the arms of the patient from becoming jammed (e.g., when the local coil array, together with the patient, is moved on a patient table). In one embodiment, the anti-trap structure prevents the patient from being able to move his arm out of the inner volume of the bearing structure.

In one embodiment, the anti-trap structure is planar. In one embodiment, the anti-trap structure extends over part of the surface left open by the recess. In one embodiment, this part makes up more than 50% (e.g., more than 70 %) of the surface left open by the recess.

In one embodiment, the anti-trap structure is elastic. In one embodiment, the anti-trap structure has a mechanical flexibility. In one embodiment, this increases the patient's comfort.

In one embodiment, the anti-trap structure is configured as a fabric (e.g., a textile fabric) and/or mesh.

In one embodiment, the bearing structure includes an adjustment mechanism. The anterior radio-frequency transmitting unit is arranged on the adjustment mechanism. The adjustment mechanism is configured to tilt the anterior radio-frequency transmitting unit (e.g., about an axis of rotation oriented perpendicular to the sagittal plane) and/or to displace the anterior radio-frequency transmitting unit (e.g., perpendicular to the frontal plane). In one embodiment, the adjustment mechanism is arranged on the upper side of the bearing structure.

For the displacement, the adjustment mechanism may include, for example, a guide system (e.g., rails). For the tilting, the adjustment mechanism may include, for example, a swivel system (e.g., swivel joint and/or pivot bearing). The swivel joint and/or pivot bearing may be arranged, for example, on a frame of the adjustment mechanism.

In one embodiment, the local coil array (e.g., the anterior part) may be better adjusted to the body shape of the patient as a result of the adjustment options. For example, a possible displacement of the radio-frequency transmitting unit in the direction of the patient or away from the patient may adjust the local coil array to the girth of the patient. In one embodiment, a possible tilting of the radio-frequency transmitting unit may adjust the local coil array to the body profile of the patient.

One possible embodiment of the local coil array provides that the local coil array includes a posterior part. The posterior part includes a posterior radio-frequency transmitting unit. The anterior part (e.g., the bearing structure) is configured to be arranged (e.g., reversibly) on the posterior part (e.g., on the posterior radio-frequency transmitting unit). In one embodiment, the anterior part may be removed from the posterior part.

In one embodiment, the posterior part may first be arranged on a patient table in order to carry out a magnetic resonance examination. The patient may then be placed on the posterior part. The anterior part may subsequently be arranged on the posterior part, so the patient (e.g., a section of torso of the patient) is located in the inner volume of the bearing structure of the anterior part.

For example, the bearing structure is configured to be arranged with its supporting side on the posterior part (e.g., on the posterior radio-frequency transmitting unit). In one embodiment, the anterior radio-frequency transmitting unit and the posterior radio-frequency transmitting unit are arranged on two opposite sides (e.g., a lower side and an upper side) of the bearing structure during a magnetic resonance examination of the section of torso of the patient. For example, the bearing structure has supporting surfaces and/or support points that are configured to be arranged on the posterior part (e.g., on the posterior radio-frequency transmitting unit). In one embodiment, the posterior part includes a support for arrangement of the anterior part. For example, the bearing structure may be placed from above onto the posterior part.

In one embodiment, the posterior part has a shape corresponding to the supporting surfaces and/or support points. In one embodiment, the shape of the posterior part is configured to produce a positive fit with the anterior part (e.g., on the supporting surfaces and/or support points of the bearing structure).

The posterior part is, for example, a lower part and/or a bottom part. In one embodiment, the posterior part (e.g., the posterior radio-frequency transmitting unit) is configured to support the section of torso of the patient thereon.

In one embodiment, the posterior part delimits the inner volume of the bearing structure when the anterior part is arranged on the posterior part (e.g., as intended). In one embodiment, the posterior part and the anterior part delimit the volume in which the section of torso of the patient may be positioned.

The posterior radio-frequency transmitting unit may include, for example, one or more transmitting antennas. For example, each transmitting antenna may be assigned to a transmission channel. If the anterior radio-frequency transmitting unit and/or the posterior radio-frequency transmitting unit together has a plurality of transmitting antennas or transmission channels, a parallel transmission (pTx) may be possible with the local coil array.

One possible embodiment of the local coil array provides that the bearing structure includes at least one anterior plug-in connector part that is arranged on the supporting side of the bearing structure. The posterior part includes at least one posterior plug-in connector part. The at least one anterior plug-in connector part and the at least one posterior plug-in connector part are configured to produce a (e.g., detachable) connection for the mechanical fixing and/or signal transmission. In one embodiment, the anterior part is latched to the posterior part of the local coil array at the same time as a connection for signal transmission.

In one embodiment, the at least one posterior plug-in connector part is a posterior plug-in connector part corresponding to the at least one anterior plug-in connector part. For example, the at least one posterior plug-in connector part acts as at least one socket connector and the at least one anterior plug-in connector part as at least one plug (e.g., corresponding thereto). For example, the at least one anterior plug-in connector part acts as at least one socket connector, and the at least one posterior plug-in connector part acts as at least one plug (e.g., corresponding thereto).

In one embodiment, the mechanical fixing brings about a mechanical fixing of the anterior part to the posterior part. In one embodiment, the mechanical fixing includes a latching of the anterior part to the posterior part. In one embodiment, the mechanical fixing is produced by a positive fit of the anterior plug-in connector part with the posterior plug-in connector part.

In one embodiment, the signal transmission includes a transmission of RF signals (e.g., transmission signals (of RF transmission pulses)) and/or receive signals (e.g., magnetic resonance signals). The signals may be transmitted, for example, electrically (e.g., using electric conductors) and/or optically (e.g., using optical conductors).

One possible embodiment of the local coil array provides that the anterior part (e.g., the bearing structure of the anterior part) and the posterior part are configured to be mechanically fixed together in exactly two different orientations. The two different orientations are, for example, two mutually opposed orientations. The two different orientations are, for example, orientations rotated about 180°. In one embodiment, these orientations are oriented perpendicular to the transversal plane (e.g., +z-direction and −z-direction).

In one embodiment, the orientation of the posterior part is strictly predefined relative to the patient table on which the patient is positioned. For example, the posterior part may be arranged (e.g., fixed and/or attached) only in a specific orientation on the patient table. In one embodiment, the orientation of the anterior part relative to the patient table may then also have exactly two different states. In one embodiment, with respect to the one state, the orientation of the other state is rotated about 180°.

In one embodiment, the posterior part includes exactly two diagonally arranged posterior plug-in connector parts and the anterior part exactly one anterior plug-in connector part, with it being possible to plug the anterior plug-in connector part in each of the two posterior plug-in connector parts and the diagonal arrangement of the posterior plug-in connector parts makes exactly two relative orientations possible (e.g., rotated by 180°) in order to plug the anterior part into the posterior part.

In one embodiment, the local coil array is configured to output information about an orientation (e.g., in a connected state) of the anterior part relative to the posterior part. For example, it is possible to derive from this information in which of the two diagonally arranged posterior plug-in connector parts, described in the preceding paragraph, the anterior plug-in connector part is plugged.

The connected state is, for example, the state in which a connection is produced between the anterior part and the posterior part. The connected state is, for example, the state in which the at least one posterior plug-in connector part is connected to the at least one anterior plug-in connector part.

A coil code, for example, of the local coil array may be read out in order to output the information about the orientation. For example, the coil code is transmitted via two lines. In one embodiment, the magnetic resonance apparatus is configured to capture an electrical resistance of the lines, via which the orientation may be ascertained. For example, the magnetic resonance apparatus is configured to assign the detected electrical resistance a, for example, hexadecimal, code (e.g., 0x0-0xF) via a look-up table.

In one embodiment, the detectable electrical resistance is dependent on the orientation of the local coil array. The electrical resistance of the anterior plug-in connector part with a foot-side arrangement has a different value than with a head-end arrangement. In one embodiment, the magnetic resonance apparatus may thereby detect whether the anterior plug-in connector part was plugged in at the foot end or the head end.

In one embodiment, the orientation of the body is strictly predefined relative orientation of the local coil array. If it is again identified in which orientation the local coil array is arranged on the patient table, then the orientation of the body may also be determined. For example, a headfirst orientation may be distinguished from a feet first orientation.

One possible embodiment of the local coil array provides that the posterior part includes at least one extension unit. The at least one extension unit is configured to lengthen an extension of the posterior part in the frontal plane (e.g., perpendicularly to the transversal plane).

In one embodiment, the extension unit includes an intermediate piece (e.g., a spacer and/or plugin location adapter) that is provided to be arranged between a (e.g., fixed) attachment point on the patient table and the posterior radio-frequency transmitting unit. In one embodiment, the extension unit may be fixed to the attachment point on the patient table and/or be connected to the patient table.

In one embodiment, the spacing between the posterior radio-frequency transmitting unit and the attachment point on the patient table may be set using the extension unit. In one embodiment, different body regions of the patient may thus be examined better with the aid of the local coil array. In one embodiment, the extension unit has a length (e.g., perpendicular to the transversal plane) of 10 to 30 cm (e.g., 20 cm). In one embodiment, an extension unit of such a length may define a second organ position (e.g., heart). If the SAR monitoring (e.g., VOP or K-factor) is configured for an axial position tolerance of, for example, ±10 cm, then organ examinations of the entire upper body may be possible.

In one embodiment, the posterior radio-frequency transmitting unit and the at least one extension unit include a (e.g., shared) local coil interface, which is configured to transmit signals (e.g., transmission signals and/or receive signals). For example, transmission signals (e.g., transmission pulses) generated by a control unit of the magnetic resonance apparatus may be transmitted via the local coil interface. For example, magnetic resonance signals received from the local coil array may be transmitted via the local coil interface to an evaluation unit of the magnetic resonance apparatus. The local coil interface may include, for example, a plug-in connection for signal transmission.

In one embodiment, the local coil interface is configured to produce a, for example, detachable connection for the mechanical fixing of the posterior radio-frequency transmitting unit to the at least one extension unit. The fixing may be effected, for example, by a positive fit (e.g., a plug-in connection for signal transmission).

In one embodiment, the local coil interface is configured to produce the connection via a movement of the posterior part relative to the extension unit perpendicular to the transversal plane.

A patient table with a local coil array as previously described is also provided, where the patient table includes a receiver for receiving the local coil array (e.g., for receiving a posterior part of the local coil array).

In one embodiment, posterior radio-frequency transmitting unit and the patient table includes a (shared) system interface that is configured to transmit signals (e.g., transmission signals and/or receive signals). For example, transmission signals (e.g., transmission pulses) generated by a control unit of the magnetic resonance apparatus may be transmitted via the system interface. For example, magnetic resonance signals received from the local coil array may be transmitted to an evaluation unit of the magnetic resonance apparatus via the system interface. The system interface may include, for example, a plug-in connection for signal transmission.

In one embodiment, the system interface is configured to produce a, for example, detachable connection for the mechanical fixing of the local coil array to the patient table. The fixing may be effected, for example, by a positive fit (e.g., a plug-in connection for signal transmission).

In one embodiment, the system interface is configured to produce the connection via a movement of the local coil array relative to the patient table perpendicular to the transversal plane.

A magnetic resonance apparatus with a local coil array as previously described is also provided. The advantages of the magnetic resonance apparatus substantially correspond to the advantages of the local coil array as previously described. Features, advantages, or alternative embodiments mentioned in this connection may likewise also be transferred to the magnetic resonance apparatus.

Further advantages, features, and details of the present embodiments may be found in the example embodiments described below, as well as based on the drawings. Mutually corresponding parts are provided with same reference numerals in all figures.

### BRIEF DESCRIPTION OF THE DRAWINGS

FIG. 1 shows a magnetic resonance apparatus with a local coil array in a schematic representation;

FIG. 2 shows anatomical planes to describe possible features of the local coil array;

FIG. 3 shows a local coil array on a patient table;

FIG. 4 shows a local coil array in a first arrangement on the patient table;

FIG. 5 shows a local coil array in a second arrangement on the patient table;

FIG. 6 shows a local coil array with an extension unit in a third arrangement on the patient table;

FIG. 7 shows a local coil array with a displaceable anterior radio-frequency transmitting unit; and

FIG. 8 shows a local coil array with a tiltable anterior radio-frequency transmitting unit.

### DETAILED DESCRIPTION

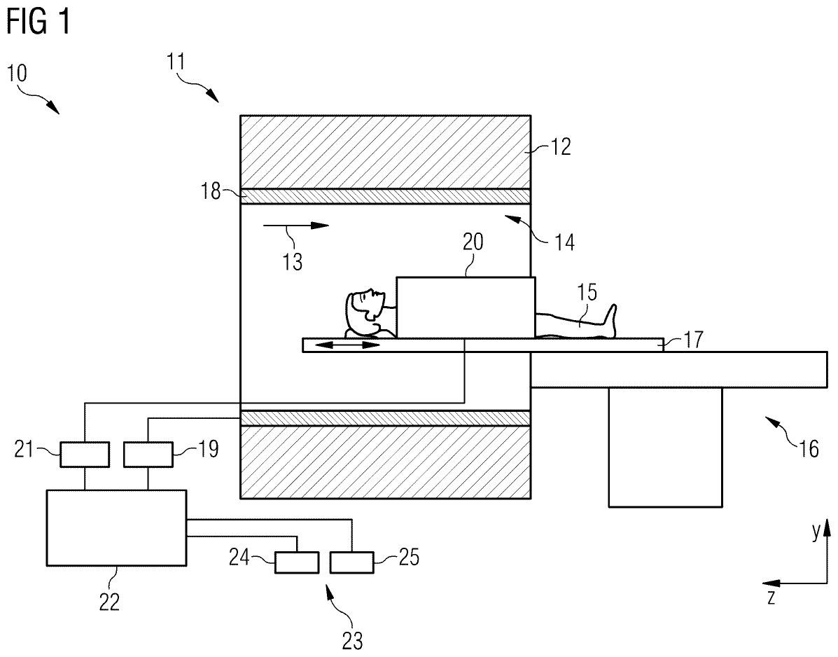

FIG. 1 schematically represents a magnetic resonance apparatus 10. The magnetic resonance apparatus 10 includes a magnet unit 11 that has a main magnet 12 for generating a strong and, for example, time-constant main magnetic field 13. In addition, the magnetic resonance apparatus 10 includes a patient-receiving region 14 for receiving a patient 15. The patient-receiving region 14 in the present example embodiment is cylindrical and is cylindrically surrounded in a circumferential direction by the magnet unit 11. In principle, however, a different embodiment of the patient-receiving region 14 may be provided. The patient 15 may be pushed into the patient-receiving region 14 by a patient-supporting apparatus 16 of the magnetic resonance apparatus 10. The patient-supporting apparatus 16 has for this purpose a patient table 17 that is configured to move (e.g., in the z-direction) inside the patient-receiving region 14.

The magnet unit 11 also has a gradient coil unit 18 for generating magnetic field gradients that are used for spatial encoding during imaging. The gradient coil unit 18 is controlled by a gradient control unit 19 of the magnetic resonance apparatus 10. The magnet unit 11 also includes a radio-frequency antenna unit 20 that in the present example embodiment is configured as a local coil array 100 represented in detail in FIGS. 3 to 8. The radio-frequency antenna unit 20 is controlled by a radio-frequency antenna control unit 21 of the magnetic resonance apparatus 10 and irradiates radio-frequency magnetic resonance sequences into an examination space that is substantially formed by a patient-receiving region 14 of the magnetic resonance apparatus 10. As a result, an excitation of atomic nuclei by the main magnetic field 13 generated by the main magnet 12 occurs. Relaxation of the excited atomic nuclei generates magnetic resonance signals. The radio-frequency antenna unit 20 may also be configured to receive the magnetic resonance signals.

The magnetic resonance apparatus 10 has a system control unit 22 in order to control the main magnet 12, the gradient control unit 19, and to control the radio-frequency antenna control unit 21. The system control unit 22 centrally controls the magnetic resonance apparatus 10, such as, for example, the implementation of a predetermined magnetic resonance system. In addition, the system control unit 22 includes an evaluation unit (not represented) for evaluating the magnetic resonance signals that are captured during the magnetic resonance examination. Further, the magnetic resonance apparatus 10 includes a user interface 23 that is connected to the system control unit 22. Items of control information, such as imaging parameters, as well as reconstructed magnetic resonance mappings, may be displayed for a medical operator on a display unit 24 (e.g., on at least one monitor) of the user interface 23. Further, the user interface 23 includes an input unit 25, by which items of information and/or parameters may be input by the medical operator during a measuring process.

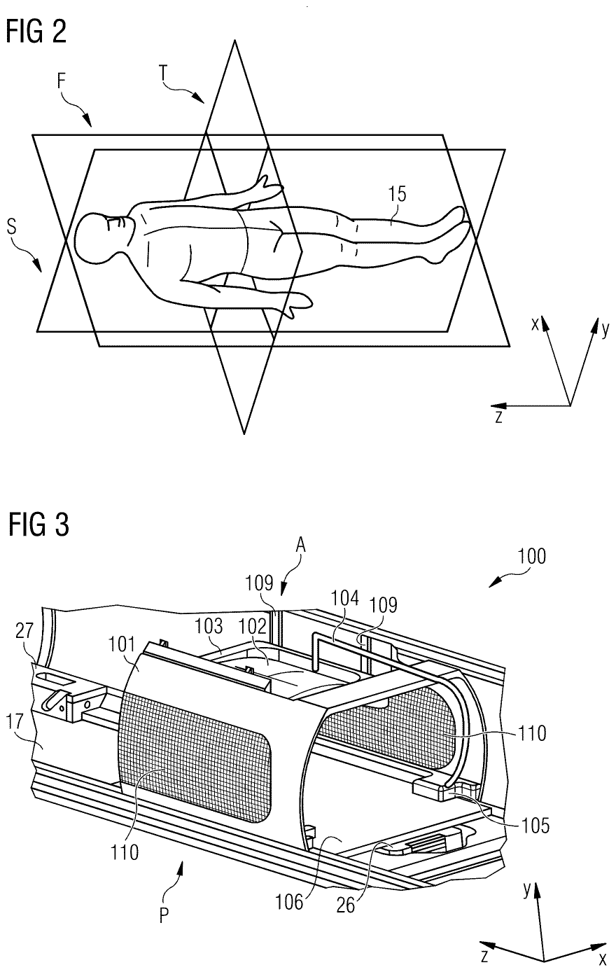

Anatomical planes of the patient 15 are to be illustrated based on FIG. 2. These also serve to describe various possible properties of the local coil array 200 described based on the following figures. It is assumed in this connection that the patient 15 is positioned in the local coil array 200 as intended (e.g., as the following figures represent; in particular, lying on their back or abdomen). The anatomical planes of the patient 15 are to be transferred to the local coil array 200 (e.g., the geometric construction of the local coil array 200) accordingly.

A distinction is made between a transversal plane T (e.g., parallel to the xy-plane), a sagittal plane S (e.g., parallel to the yz-plane), and a frontal plane F (e.g., parallel to the xz-plane). All of these planes are oriented perpendicular to one another. The transversal plane T is oriented perpendicular to the longitudinal axis of the patient 15. The transversal plane T divides the body of the patient into a lower side (e.g., including his legs) and an upper side (e.g., including his upper body). The sagittal plane divides the body of the patient into a left and a right half of the body. The frontal plane F is parallel to the surface on which the patient 15 is lying. The frontal plane F divides the body of the patient 15 into a front side and a rear side.

FIG. 3 shows a local coil array 100 for magnetic resonance examination of a section of torso of the patient 15, with an anterior part A and a posterior part P. The posterior part P includes a posterior radio-frequency transmitting unit 106 and is arranged on the patient table 17. The two coil parts A, B may form a mechanical and electrical unit, enabling simple and reproducible patient positioning. It is simultaneously provided that the relative position of local coil array 100 and patient 15 remains within predictable limits. Within these limits, SAR simulations with human model and local coil array 100 may specify safe dose values. This is important, for example, for an application of a free pTx method to be able to estimate SAR hotspots for free transmission phases and amplitudes as accurately as possible.

The posterior part P (e.g., the posterior radio-frequency transmitting unit 106) is connected to the magnetic resonance apparatus 10 via a system interface 26. For example, signals may be transmitted from the magnetic resonance apparatus 10 to the local coil array 100 and/or from the local coil array 100 to the magnetic resonance apparatus 10 via the system interface 26. In addition, the local coil array 100 may be latched to the patient table 17 using the system interface 26. Such a direct plugging of the local coil array 100 to the patient table enables a standardized clinical workflow (e.g., in contrast to any plugging-in using freely movable cables).

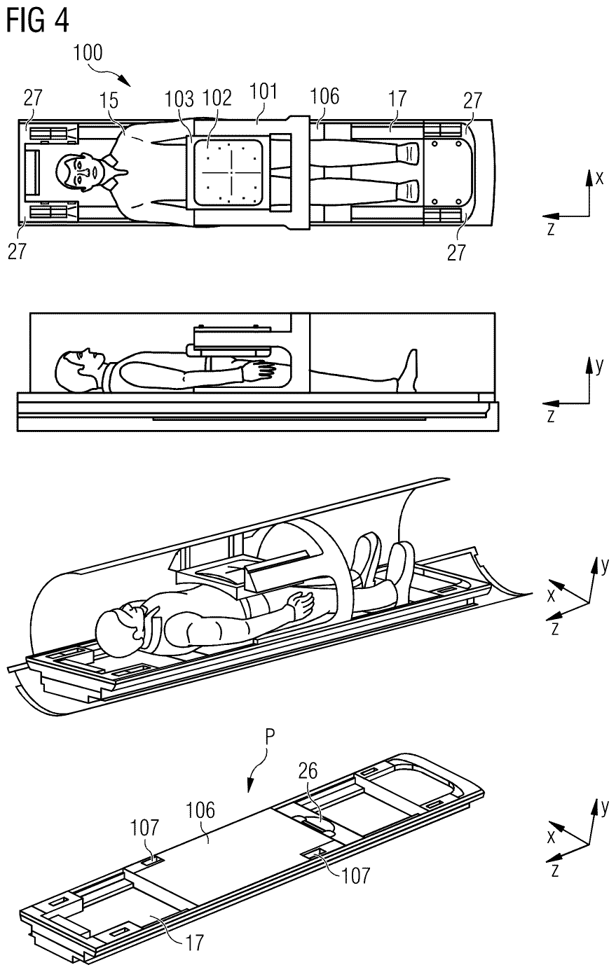

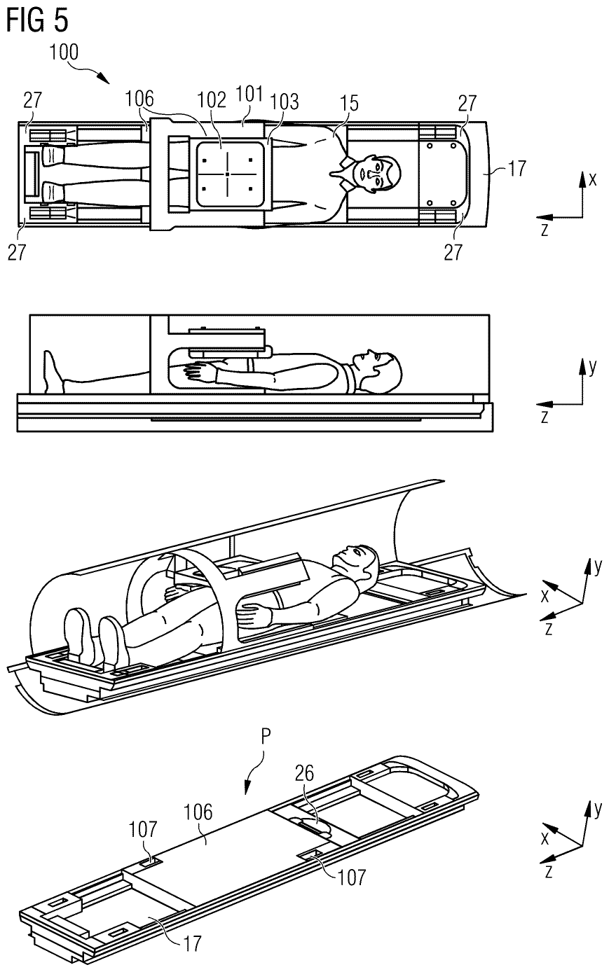

The anterior part A includes a bearing structure 101 and an anterior radio-frequency transmitting unit 102, which is arranged on the upper side of the bearing structure 101. The bearing structure 101 is detachably connected to the posterior part P on the supporting side of the bearing structure 101. The bearing structure 101 has an inner volume in which the section of torso of the patient 15 to be examined may be positioned. Such a positioning of the patient 15 in the inner volume of the bearing structure 101 is represented by way of example in FIGS. 4 to 6, which respectively show (from top to bottom) a plan view, a side view, a perspective view of a local coil array 100 with a patient 15 positioned therein, as well as a perspective view of the posterior part P.

The bearing structure 101 has an elongate recess on either side, to or in which an anti-trap structure 110 in the form of a mesh or fabric is fastened. The anti-trap structure 110 extends (e.g., substantially) parallel to the sagittal plane S. In one embodiment, the bearing structure 101 may prevent injuries (e.g., crush injuries) occurring when the patient 15 on the patient table 17 is moved through the magnet unit 11 of the magnetic resonance apparatus 10.

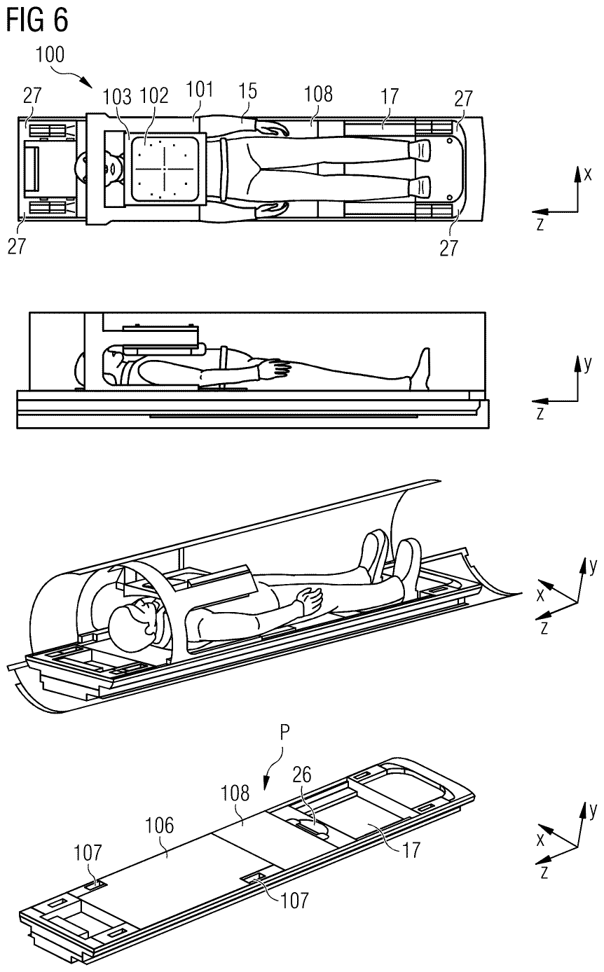

The section of torso to be examined is the pelvic area according to FIGS. 4 and 5. According to FIG. 6, the chest area is examined as the section of torso. The patient 15 is supported on the patient table 17, with the section of torso to be examined being supported on the posterior radio-frequency transmitting unit 106.

The bearing structure 101 includes an anterior plug-in connector part 105 that is arranged on the supporting side of the bearing structure 101. The plug-in connector part 105 is connected (e.g., electrically) to the anterior radio-frequency transmitting unit 102 by a line 104.

The posterior part P includes two posterior plug-in connector parts 107, corresponding to the anterior plug-in connector part 105, which are shown in FIGS. 4 to 6. The anterior plug-in connector part 105 is plugged into one of the two posterior plug-in connector parts 107 in order to produce a connection for mechanical fixing and/or signal transmission.

The two posterior plug-in connector part 107 are arranged at diagonally opposite corners of the posterior radio-frequency transmitting unit 106. As a result, the bearing structure 101 may be mechanically fixed to the posterior part P in exactly two different orientations. This is represented, by way of example, in FIGS. 4 and 5. In FIG. 4, the recess of the lateral section of the bearing structure 101 is open in the +z-direction; in FIG. 5, the recess of the lateral section of the bearing structure 101 is open in the −z-direction (e.g., rotated by 180°). In one embodiment, it is thereby also possible to detect the position (e.g., head first/feet first) when the anterior part A is attached to the posterior part P.

In FIG. 6, the posterior part P includes an extension unit 108. The posterior part P may thereby be lengthened in the z-direction (e.g., the extension of the posterior part P is lengthened perpendicularly to the transversal plane T). Since the system interface 26 is fixedly arranged on the patient table, the posterior radio-frequency transmitting unit 106 is displaced by the length of the extension unit 108 perpendicular to the transversal plane T (e.g., in the +z-direction). When the position of the patient 15 on the patient table remains the same, the radio-frequency transmitting units 106, 107 of the local coil array 100 are displaced to a different section of torso of the patient 15. According to FIG. 5, in the case without lengthening, the radio-frequency transmitting units 106, 107 are thus located in the pelvic area of the patient 15, while the radio-frequency transmitting units 106, 107 are located in the chest area with a lengthening according to FIG. 6.

The bearing structure 101 is rigid, so the bearing structure 101 may safely support the anterior radio-frequency transmitting unit 102. At its upper side, the bearing structure 101 includes a frame 103 on which the anterior radio-frequency transmitting unit 102 is arranged. The bearing structure has guides 109 in which the frame 103 may be displaced vertically (e.g., perpendicularly to the frontal plane). The bearing structure 101 includes a lateral section to the right and left, respectively. When the sagital plane S is viewed from the side, the lateral sections have a C-shape respectively. As shown in FIGS. 4 to 6, the C-shape of the bearing structure 101 is characterized by an elongate recess in which an arm and/or a shoulder of the patient 15 may be at least partially positioned. In one embodiment, the double C-arm structure does not result in an unnecessary restriction of the freedom of movement of shoulder, arms, or upper body for the patient 15.

The inner volume of the bearing structure is delimited from below by the posterior radio-frequency transmitting unit 106, laterally by the lateral sections of the bearing structure 101, and from above by the anterior radio-frequency transmitting unit 102.

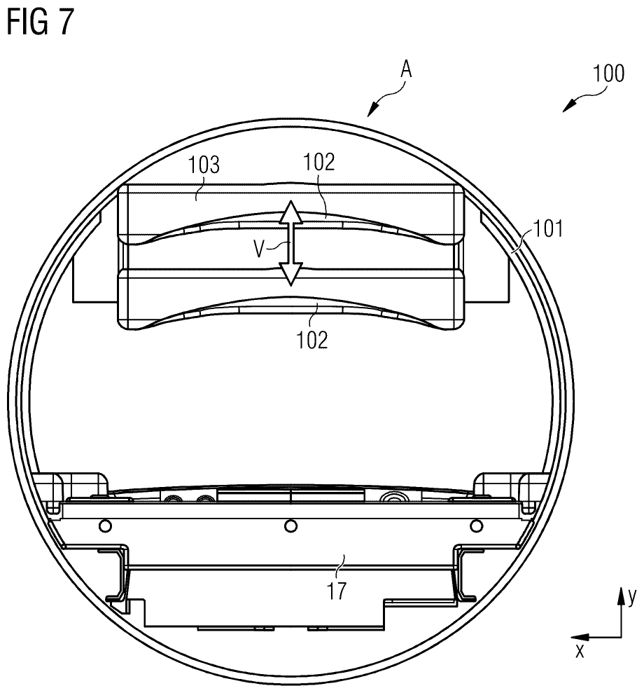

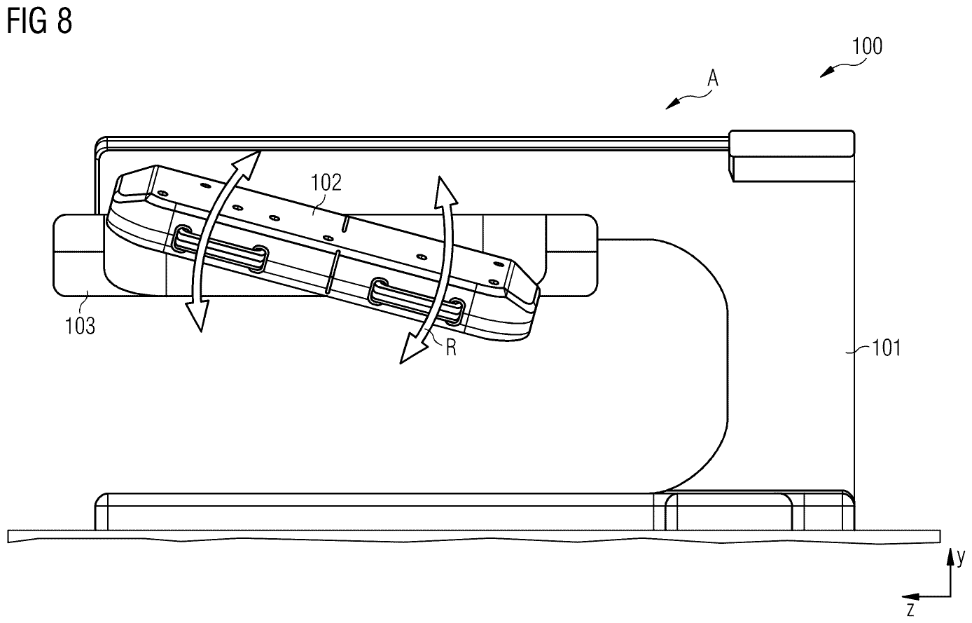

The frame 103 is part of an adjustment mechanism of the bearing structure 101. With the aid of the adjustment mechanism, it is possible to tilt the anterior radio-frequency transmitting unit 102 (e.g., about an axis of rotation oriented perpendicular to the sagittal plane S) and/or displace the anterior radio-frequency transmitting unit 102 (e.g., perpendicularly to the frontal plane F). This is represented in detail in FIGS. 7 and 8.

To enable a displacement V according to FIG. 7, the bearing structure 101 includes a linear guide, shown in FIG. 3, in which the frame 102, together with the anterior radio-frequency transmitting unit 102, may be vertically displaced.

To enable tilting R according to FIG. 8, the anterior part A includes a swivel joint between the frame 103 and the anterior radio-frequency unit 102, about which joint a rotation may be executed. The inclination of the anterior radio-frequency unit 102 may be purposefully set thereby.

The local coil array 100 may improve the clinical use of a magnetic resonance examination of the human torso. Previous solutions are often simple and flexible but simultaneously have a complex handling operation and lack of reproducibility. This is a problem for clinical serial examinations, as well as for reliable (and simultaneously high-performance) SAR monitoring. The proposed local coil array 100 may be easy to handle since, together with the coil upper part (e.g., with, adjustable height and inclination) and the coil lower part (e.g., by plug-in connection and positive fit), the local coil array 100 forms a unit. In addition, any complications due to free cables may be avoided due to the guidance of the line 104 inside the bearing structure 101. The introduction of two plug-in possibilities makes it possible to comfortably position the patient 15 both in the “feet first” and in the “head first” position. The posterior radio-frequency transmitting unit 106 retains its orientation relative to the magnet unit 11, whereas the anterior radio-frequency transmitting unit 102, together with the bearing structure 101, is rotated about 180°. As a result, it is simultaneously possible to detect which positioning was selected (e.g., mechanical and/or electrical encoding of the plug-in positions) and to transmit this information to the system control unit 22 (e.g., via coil code line, which provides individual coil parameters for the respective position, such as transmission phases of the coil elements).

In conclusion, reference is once again made to the fact that the local coil array 100 or the magnetic resonance apparatus 10 described in detail above are example embodiments that may be modified in a wide variety of ways by a person skilled in the art without departing from the field of the invention. Further, use of the indefinite article “a” or “an” does not preclude the relevant features from also being present multiple times. Similarly, the term “unit” does not preclude the relevant components from consisting of a plurality of interacting sub-components, which may possibly also be spatially distributed. Independent of the grammatical term usage, individuals with male, female, or other gender identities are included within the term.

The elements and features recited in the appended claims may be combined in different ways to produce new claims that likewise fall within the scope of the present invention. Thus, whereas the dependent claims appended below depend from only a single independent or dependent claim, it is to be understood that these dependent claims may, alternatively, be made to depend in the alternative from any preceding or following claim, whether independent or dependent. Such new combinations are to be understood as forming a part of the present specification.

While the present invention has been described above by reference to various embodiments, it should be understood that many changes and modifications can be made to the described embodiments. It is therefore intended that the foregoing description be regarded as illustrative rather than limiting, and that it be understood that all equivalents and/or combinations of embodiments are intended to be included in this description.

1. A local coil array for a magnetic resonance examination of a section of a torso of a patient, the local coil array comprising:
an anterior part that comprises a bearing structure and an anterior radio-frequency transmitting unit,
wherein the bearing structure has a supporting side and an upper side,
wherein the anterior radio-frequency transmitting unit is arranged on the upper side of the bearing structure,
wherein the bearing structure has an inner volume, and
wherein the torso of the patient is positionable within the inner volume of the bearing structure.

2. The local coil array of claim 1, wherein the bearing structure is rigid.

3. The local coil array of claim 1, wherein the bearing structure has a C-shape in a side view on a sagittal plane.

4. The local coil array of claim 3, wherein lateral sections of the bearing structure have a C-shape in the side view on the sagittal plane.

5. The local coil array of claim 3, wherein the C-shape of the bearing structure is characterized by a recess configured for positioning an arm, a shoulder, or the arm and the shoulder of the patient at least partially in the recess.

6. The local coil array of claim 5, wherein an elastic, planar, or elastic and planar anti-trap structure is arranged on the recess.

7. The local coil array of claim 1, wherein the bearing structure comprises an adjustment mechanism,
wherein the anterior radio-frequency transmitting unit is arranged on the adjustment mechanism,
wherein the adjustment mechanism is configured to tilt the anterior radio-frequency transmitting unit about an axis of rotation oriented perpendicular to the sagittal plane, displace the anterior radio-frequency transmitting unit perpendicular to the frontal plane, or a combination thereof.

8. The local coil array of claim 1, further comprising a posterior part,
wherein the posterior part comprises a posterior radio-frequency transmitting unit,
wherein the bearing structure is configured to be arranged on the posterior radio-frequency transmitting unit.

9. The local coil array of claim 8, wherein the posterior radio-frequency transmitting unit is configured to support the section of the torso of the patient on the posterior radio-frequency transmitting unit.

10. The local coil array of claim 8, wherein the bearing structure comprises at least one anterior plug-in connector part that is arranged on the supporting side of the bearing structure,
wherein the posterior part comprises at least one posterior plug-in connector part, and
wherein the at least one anterior plug-in connector part and the at least one posterior plug-in connector part are configured to produce a connection to a mechanical fixing, signal transmission, or a combination thereof.

11. The local coil array of claim 8, wherein the bearing structure and the posterior part are configured to be mechanically fixed together in exactly two different orientations.

12. The local coil array of claim 8, wherein the local coil array is configured to output information about an orientation of the anterior part relative to the posterior part.

13. The local coil array of claim 8, wherein the posterior part comprises at least one extension unit,
wherein the at least one extension unit is configured to lengthen an extension of the posterior part perpendicularly to a transversal plane, and
wherein the posterior radio-frequency transmitting unit and the at least one extension unit comprise a local coil interface configured to transmit signals.

14. A patient table comprising:
a local coil array for a magnetic resonance examination of a section of a torso of a patient, the local coil array comprising:
an anterior part that comprises a bearing structure and an anterior radio-frequency transmitting unit,
wherein the bearing structure has a supporting side and an upper side,
wherein the anterior radio-frequency transmitting unit is arranged on the upper side of the bearing structure,
wherein the bearing structure has an inner volume,
wherein the torso of the patient is positionable within the inner volume of the bearing structure, and
wherein the patient table comprises a receiver for receiving the local coil array.

15. The patient table of claim 14, wherein the receiver is configured to receive a posterior part of the local coil array.

16. The patient table of claim 14, wherein the posterior radio-frequency transmitting unit and the patient table comprise a system interface configured to transmit signals.

17. A magnetic resonance apparatus comprising:
at least one local coil array, a local coil array of the at least one local coil array being for a magnetic resonance examination of a section of a torso of a patient, the local coil array comprising:
an anterior part that comprises a bearing structure and an anterior radio-frequency transmitting unit, wherein the bearing structure has a supporting side and an upper side, wherein the anterior radio-frequency transmitting unit is arranged on the upper side of the bearing structure, wherein the bearing structure has an inner volume, and wherein the torso of the patient is positionable within the inner volume of the bearing structure;
a patient table comprising:
the local coil array; and
a receiver for receiving the local coil array; or
or a combination thereof.
